(19) United States
(12) Patent Application Publication
(10) Pub. No.: US 2019/0240442 A1
(43) Pub. Date: Aug. 8, 2019

(54) OXYGEN TUBE SAFETY SYSTEM

(71) Applicant: Terrence Murphy, Street, MD (US)

(72) Inventor: Terrence Murphy, Street, MD (US)

(21) Appl. No.: 16/269,977

(22) Filed: Feb. 7, 2019

Related U.S. Application Data
(60) Provisional application No. 62/627,246, filed on Feb. 7, 2018.

(51) Int. Cl. A61M 16/08; A61M 25/02

(52) U.S. Cl. CPC A61M 16/0875; A61M 16/0003; A61M 25/02

(57) ABSTRACT

An oxygen tube system that includes an upper-arm cuff portion with a tube collar affixed there to direct an oxygen tube from a patient's nasal cannula to their side, thereby reorienting the tube away from the feet. The arm cuff also includes a detachable fall detection medical alert transponder for alerting authorities in case there is a fall. In addition, a braided slit-tube sleeve with shape-memory is inserted over the oxygen tubes to prevent tangling and to impart an increased frictional resistance at the tube collar. Two sections of the braided slit-tube sleeve are inserted over the oxygen tubes in advance and arrears of a pivot coupling, which in combination prevent tangling of the tube, reorient and maintain the oxygen tube to the side and back of the patient away from the feet, and which ultimately detects a fall if one should occur.

### CROSS-REFERENCE TO RELATED APPLICATION(S)

The present application derives priority from U.S. provisional patent application No. 62/627,246 filed Feb. 7, 2018.

### BACKGROUND OF THE INVENTION

### 1. Field of the Invention

The present invention relates generally to safety equipment and, more particularly, to a safety system for patients that use oxygen concentrators that is designed to keep the oxygen tube running from the nasal cannula untangled and directed away from the patient's feet and behind them, so as to prevent trip and falls.

### 2. Description of the Background

Oxygen concentrators typically use pressure swing adsorption (PSA) technology to provide an economical source of oxygen to patients with breathing problems. A conventional oxygen concentrator has an air compressor, two cylinders filled with zeolite pellets, a pressure equalizing reservoir, valves, and oxygen tubes running to the patient's nose cannula.

Patients that rely on oxygen concentrators are prone to tripping and falling due to the tangle of tubing. Sometimes the run of tubing is quite long (20-50 feet or more) and as the patient ambulates about their home it becomes tangled. In this case the tubing typically runs directly downward from the nose cannula to a coiled tangle at the patient's feet, which drastically increases the risk of falling and injury. To prevent falls, oxygen medical suppliers suggest several solutions, such as placing bright colored tape along the tubing to alert the patient to its location, and holding the tubing in hand so the tube does not get tangled around the feet. The efficacy of such precautions depends on the foresight and/or memory of the patient, which is not often perfect. What is needed is an oxygen tube safety harness that works independently and more effectively, so long as it is used.

### SUMMARY OF THE INVENTION

It is an object of the present invention to provide an oxygen tube safety system that reorients and maintains the oxygen tube to the side and back of the patient, away from the feet.

It is another object of the present invention to provide an oxygen tube safety system that is comfortable and easy to put on and/or remove.

It is another object of the present invention to provide an oxygen tube safety system that prevents tangling of the tubes when the patient walks.

In accordance with the foregoing objects, the invention is an improved oxygen tube system that includes an upper-arm cuff portion to direct the oxygen tube from the patient's nasal cannula to their side, thereby reorienting the tube away from the feet. The arm cuff includes a tube collar for slidably passing the oxygen tube there through, and thereby allowing movement of the arm. The arm cuff also includes a detachable fall detection medical alert transponder for alerting authorities in case there is a fall. In addition, a braided slit-tube sleeve is inserted over the oxygen tubes to prevent tangling on one hand, and impart increased frictional resistance at the tube collar. Two sections of the braided slit-tube sleeve are inserted over the oxygen tubes in advance and arrears of a pivot coupling, which in combination with the tube sleeve sections prevents tangling of the tube despite ambulation of the patient. The system components work together as a whole to prevent tangling of the tube in the first instance, to reorient and maintain the oxygen tube to the side and back of the patient away from the feet, and to detect a fall if one should occur. Moreover, the harness is comfortable and easily removable.

### BRIEF DESCRIPTION OF THE DRAWINGS

Other objects, features, and advantages of the present invention will become more apparent from the following detailed description of the preferred embodiments and certain modifications thereof when taken together with the accompanying drawings in which:

FIG. 1 shows a front perspective view of the oxygen tube safety system 2 in use according to an embodiment of the invention.

FIG. 2 shows a side perspective view of the oxygen tube safety system 2 of FIG. 1.

FIG. 3 is a front close-up view of the arm cuff 10 portion of the oxygen tube safety system 2 of FIGS. 1-2 closed (left) and open (right inset).

FIG. 4 is a front close-up view of the tube sleeve portions 16A, 16B and pivot coupling 18 used in the oxygen tube safety system 2 of FIGS. 1-2.

### DETAILED DESCRIPTION OF THE PREFERRED EMBODIMENT

The present invention is an improved oxygen tube safety system that combines several components all of which work together as a harness to prevent tangling of the tube, and reorient and maintain the oxygen tube to the side and back of the patient away from the feet. The system also includes a fall detector just in case a fall should occur.

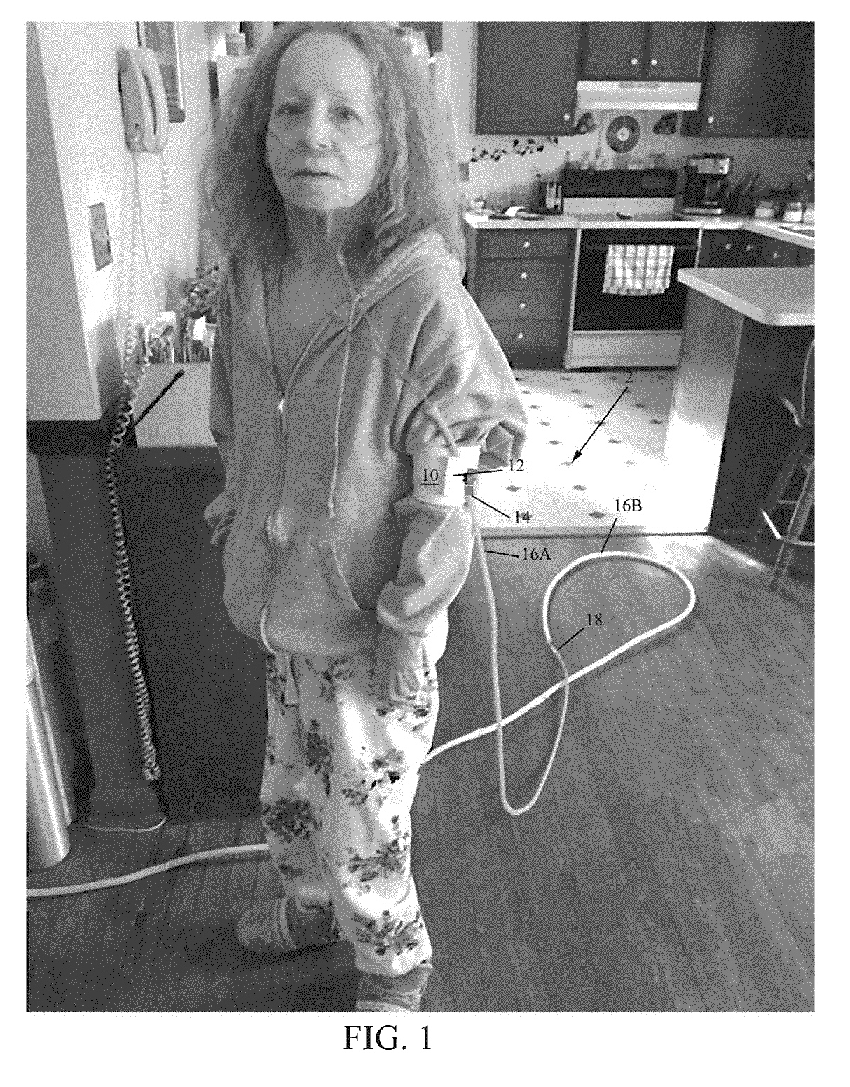

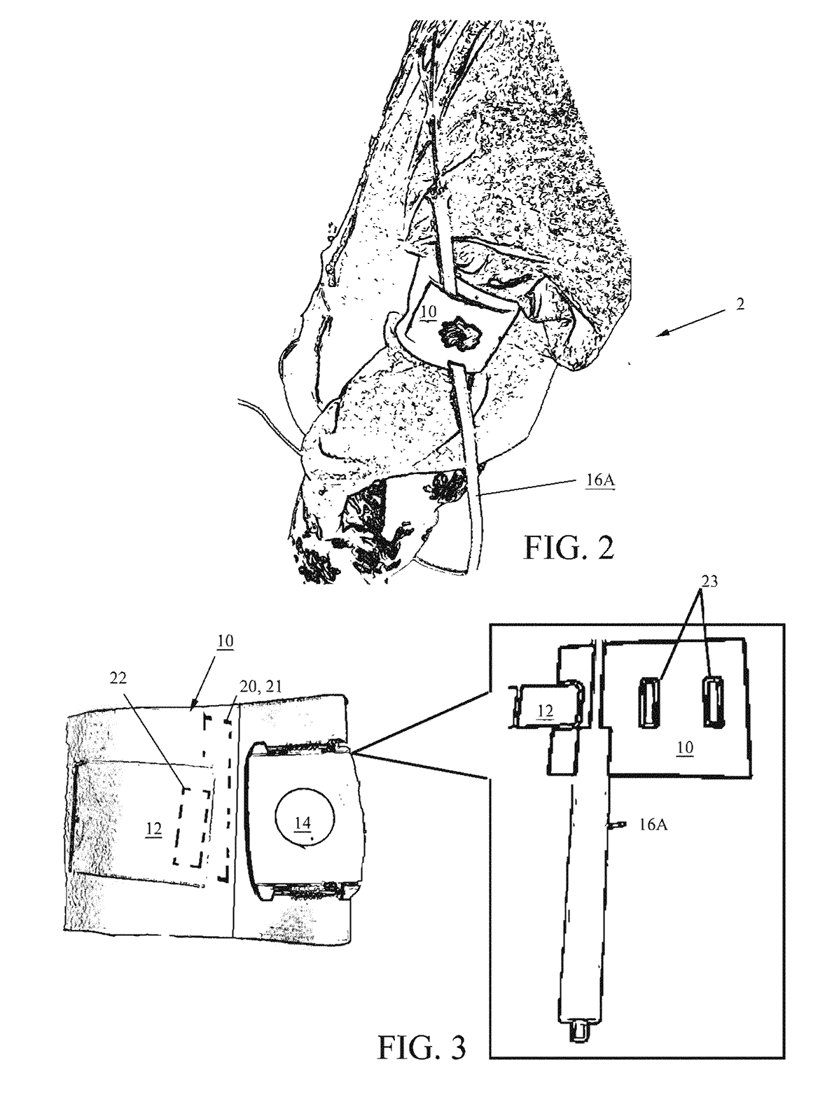

FIG. 1 shows a front perspective view of the oxygen tube safety system 2 in use, and FIG. 2 shows a side perspective view. With combined reference to FIGS. 1-2, the oxygen tube safety system 2 generally includes an upper-arm cuff portion 10 to direct the oxygen tube from the patient's nasal cannula off to their side, thereby reorienting the tube away from the feet and around back. The arm cuff 10 includes a tube collar 12 that imparts a moderated degree of friction but slidably passes the oxygen tube there through, allowing movement of the arm. The arm cuff 10 also includes a detachable fall detection medical alert transponder 14 for alerting authorities in case there is a fall. In addition, a braided slit-tube sleeve 16 is inserted over the oxygen tubes to increase rigidity and thereby prevent tangling, and to increase the coefficient of friction resistance at the tube collar 12. Two sections 16A, 16B of the braided slit-tube sleeve 16A, 16B are inserted over the oxygen tubes, section 16A in advance and 16B in arrears of a pivot coupling 18. The pivot coupling 18 in combination with the two tube sleeve sections 16A, 16B prevents tangling of the tube despite ambulation of the patient about the home. All the foregoing system components work together as a whole to 1) prevent tangling of the tube in the first place; 2) reorient and maintain the oxygen tube to the side and back of the patient away from the feet; and 3) detect a fall if one should occur. Moreover, the harness system 2 is comfortable and easily removable. Each component will herein be described in detail.

FIG. 3 is a front close-up view of the arm cuff 10 portion of the oxygen tube safety system 2 of FIGS. 1-2 closed (left) and open (right inset). Arm cuff 10 generally comprises a 2-4″ wide elasticized knit fabric band in various colors, with distal hook-or-loop attachment pads 20, 21 for fixation onto itself around the arm of the patient. The length of arm cuff 10 may vary according to arm size, from a size XXS of about 7.5″ to XXXL of about 16.75.″

As seen in the right inset, a corresponding series of one, two, three or more hook-or-loop attachment pads 23 may be spaced along cuff 10 for adjusting the size of cuff 10 by variable attachment of distal hook-or-loop attachment pads 20, 21. The cuff 10 is stretchable and can be made of Lycra® to accommodate various arm sizes. A smaller strip of stretchable fabric 12 is fixedly attached at one end to cuff 10 and bears a distal hook attachment pad 22 on its inside distal end for attachment to cuff 10, thereby forming a releasable collar 12. Collar 12 may be opened as shown at right to place the tubing and secure it underneath. Collar 12 may be closed as shown at left to secure the tubing underneath, yet still allowing it to slide/float to accommodate arm movement and avoid hindering the patient.

The tube collar 12 constrains sliding of the oxygen tube there through by imparting a moderated degree of friction. This is accomplished with a particular fabric structure of collar 12, and specifically by controlling the surface characteristics, weave, type of fabric and elastic bland proportion of collar 12 relative to sleeve 16 (below) to produce a static frictional coefficient value of between ⅓ and 1, most preferably about 0.8.

Component 14 is a medical alert monitoring pendant with button that the patient must press in order to call for help. A variety of medical alert pendants 14 are commercially available, and pendant 14 is preferably attached by hook and loop to cuff 10 to allow the patient to slap it into place on the arm as another safety option in case of fall.

In addition to the foregoing, there is another connection to the cuff 10 that provides added protection and reduces the amount of twist and coils in the tube. Two discrete sections of braided slit sleeve 16A, 16B are inserted over the oxygen tube on opposing sides of a pivot coupling 18. Pivot coupling 18 is preferably a swiveling oxygen supply tubing connector with standard 5 mm to 7 mm male end fittings such as is commercially available from Salter Labs®.

Pivot coupling 18 is preferably inserted into the oxygen supply line within a range of from 5-8 feet from the nasal cannula. A first section of braided slit sleeve 16A is inserted over the oxygen tube so as to cover the tube from 2-3 inches above the cuff 10 all the way to the pivot coupling 18, e.g., approximately 4-7 feet. A second section of braided slit sleeve 16B is inserted over the oxygen tube running from pivot coupling 18 to the oxygen concentrator.

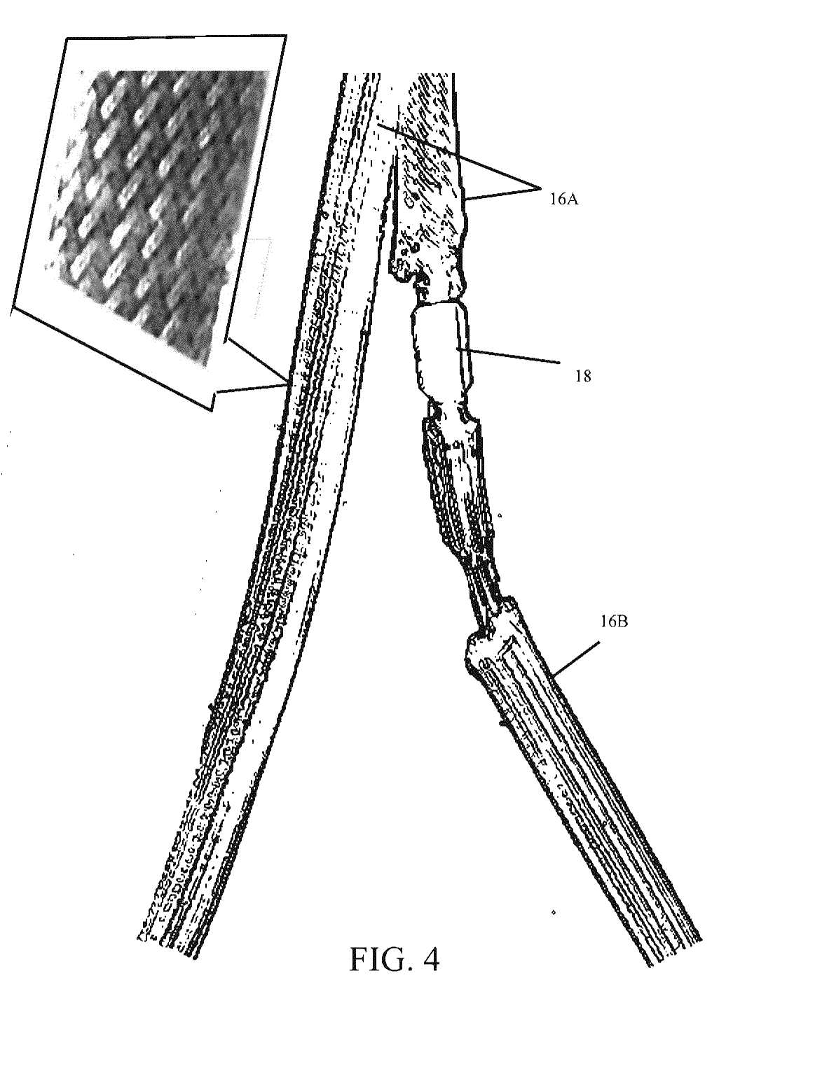

FIG. 4 is a front close-up view of the tube sleeve portions 16A, 16B and pivot coupling 18 used in the oxygen tube safety system 2 of FIGS. 1-2.

As seen in the left inset to FIG. 4, both tube sleeve portions 16A, 16B preferably comprises a woven fabric layer formed of an insulating, heat resistant yarn, such as glass fiber, relatively tightly interlaced as by weaving, braiding or knitting. A preferred material for cover layer is E-glass yarn, a natural, lustrous, white, continuous filament yarn of high stability and durability that meets the certification for E glass as defined by ASTM's D578-00 Standard Specification for Glass Fiber Strands. The E-glass yarn is preferably tightly woven and is impregnated with a high temperature coating material, such as epoxy/silicone resin, which eliminates end fray and provides resistance to abrading of the glass fibers. The yarn is tubular woven, impregnated, and solidified then slitted lengthwise to retain a tubular shape memory characteristic. Both tube sleeve portions 16A, 16B so formed are readily bendable yet impart both stiffness and a friction reducing characteristic when wrapped around the rubber oxygen tube. The axial slit facilitates easy insertion onto the tube and will accommodate a range of diameters. The sleeves 16A, 16B are easily cut to length and installed without the need for special tools.

Importantly, tube sleeve portion 16A extends through and past collar 12 by a few inches. This places imparts a degree of additional increased frictional resistance at the tube collar 12 junction by virtue of the woven sleeve 16A to fabric interface. This increased frictional resistance at the tube collar 12 attempts to maintain the proper lengths of oxygen tube between collar 12 and nasal cannula and between collar 12 and pivot coupling 18, and provides tactile feedback to the patient in case of slippage, so that proper lengths can be readjusted to avoid tangling.

All the foregoing system components work together as a whole to prevent tangling of the oxygen tube, the cuff 10 reorienting and maintain the oxygen tube to the side and back of the patient away from the feet, the combined sleeves 16A, 16B and pivot coupling 18 avoiding tangles, and the pendant 14 detecting a fall if one should occur. Moreover, the harness is comfortable and easily removable.

One skilled in the art will readily understand that other features may be implemented without departing from the scope of spirit of the invention. All such changes, modifications, variations and other uses and applications which do not depart from the spirit and scope of the invention are deemed to be sectioned by the invention, which is to be limited only by the claims which follow.

I claim:

1. A safety system for use in combination with an oxygen concentrator having a nasal cannula connected by an oxygen tube, a safety system to prevent tangling of said oxygen tube and away from a patient's feet, comprising:
an upper-arm cuff configured to be worn around said patient's forearm;
a releasable tube collar attached to said upper arm cuff for constraining said oxygen tube, said releasable tube collar having a releasable fastener for securement to itself around said oxygen tube;
a braided slit-tube sleeve enveloping said oxygen tube;
whereby a coefficient of friction of said releasable tube collar against said braided slit tube sleeve moderates sliding of said oxygen tube through said releasable tube collar when secured to itself around said oxygen tube.

2. The safety system of claim 1, wherein said oxygen tube has an intermediate pivot coupling and said braided slit-tube sleeve enveloping said oxygen tube comprises a first section on one side of said pivot coupling and a second section on another side of said pivot coupling.

3. The safety system of claim 1, wherein said arm cuff comprises a fabric band with distal hook-or-loop attachment pads for fixation onto itself around the forearm of the patient.

4. The safety system of claim 3, wherein said arm cuff comprises an elasticized knit fabric band.

5. The safety system of claim 1, wherein said tube collar and said braided slit-tube sleeve have a relative static frictional coefficient value above ¼.

6. The safety system of claim 5, wherein said tube collar and said braided slit-tube sleeve have a relative static frictional coefficient value within a range of between ⅓ and 1.

7. The safety system of claim 6, wherein said tube collar and said braided slit-tube sleeve have a relative static frictional coefficient value of approximately 0.8.

8. The safety system of claim 5, wherein said braided slit-tube sleeve comprises a woven heat-resistant fiber.

9. The safety system of claim 8, wherein said braided slit-tube sleeve comprises woven glass fiber.

10. The safety system of claim 1, further comprising a fall detection medical alert transponder attached to said upper-arm cuff.

11. The safety system of claim 10, wherein said fall detection medical alert transponder is detachable from said upper-arm cuff.

12. The safety system of claim 5, wherein said braided slit-tube sleeve has a shape-memory characteristic.

13. The safety system of claim 1, wherein said braided slit-tube sleeve has a lengthwise axial slit.
